(19) United States
(12) Patent Application Publication
(10) Pub. No.: US 2002/0153007 A1
(43) Pub. Date: Oct. 24, 2002

(54) AROMA DELIVERY DEVICE

(76) Inventor: RICHARD A. DAVI, GLENWOOD, NJ (US)

Correspondence Address: NEAL L SLIFKIN ESQ, HARRIS BEACH & WILCOX LLP, 130 EAST MAIN STREET, ROCHESTER, NY, 146041687

(21) Appl. No.: 09/406,929

(22) Filed: Sep. 28, 1999

(51) Int. Cl. A62B 7/00; A62B 9/00; A61M 15/00; A61M 16/00; A62B 18/00; A61M 16/10

(52) U.S. Cl. 128/200.24

(57) ABSTRACT

A horseshoe-shaped clip formed of a non-volatile elastomeric resin combined with one or more volatile aromatic materials, the clip being comfortably, easily, and releasably applied to the cartilaginous septum of a wearer's nose, whereby aromatic material continuously given off by the clip is entrained in the nasal inhalations of a wearer and conveyed thereby to the wearer's olfactory sensors to create a desired aromatic sensation. The device is useful in administering aromatherapy. Preferably, the aromatic material is applied to the surface of resin beads prior to injection molding of the clip. The clip is thereby permeated throughout by the aromatic material such that emission of the material is virtually undiminished over long periods of use, and the clip may be cleaned, washed, reshaped, or resurfaced. An attachable clip supplement including a second aromatic material may be provided to afford a user a choice of combinations of secondary aromas with the primary clip aroma.

### DESCRIPTION

The present invention relates to apparatus for delivering aromatic materials to olfactory receptors in the nose, more particularly to odoriferous apparatus for insertion into the nose, and most particularly to a semi-rigid, flexible nose clip, incorporating one or more aromatic materials, for removable retention in the nose to provide a continuous supply of the aromatic material to the interior of the nose. More generally, the present invention relates to means for directly, continuously, and exclusively administering aromatherapy to an individual person.

Aromatic material or chemical compound, as used herein, refers to any material or compound capable of existing in a gaseous or vapor state and, while in this state, being received in olfactory receptors of the nose, thereby to create a sensation of an aroma in the brain. (The terms aroma, odor, fragrance, and smell may be used herein interchangeably to refer to the sensation produced.) Thus, “aromatic” is an objective descriptor of a material or chemical compound, regardless of whether an aroma is actually being produced at any given time.

It is known that the mental state of a person may be influenced and altered by exposure of that person's olfactory system to any of a myriad of aromatic materials, a fact highly useful to, for example, perfume manufacturers, chefs, and skunks. Further, it is believed by many persons that some types of olfactory alteration of the mental state can give rise to alteration of the physical state, for example, to promote or accelerate healing.

Aromatic materials have been used in folk art and medicine for many years. In the early part of the 20th century, the French chemist Rene-Maurice Gattefosse coined the term “aromatherapy” to connote the use of aromas to promote mental and physical well-being. Many such materials, especially so-called “essential oils” derived from a wide variety of plants, or their synthetic counterparts, are commonly used today in aromatheraphy to relieve specific mental and physical distress; for example, oil of rose is said to relieve anger, a mixture of oil of lavender, geranium, ylang-ylang, bergamot, and melissa is said to relieve anxiety, oil of rosemary and basil or mint and jasmine is said to relieve drowsiness, and oil of lavender is said to relieve jet lag and panic attack; while oil of peppermint is said to relieve headache, oil of peppermint and nutmeg or lavender and cypress is said to relieve menstrual cramps, and oil of chamomile and lemon is said to relieve hay fever.

Suitable aromatic materials can be extremely expensive, for example, a five-mililitre bottle of oil of citrus, lavender, peppermint, rosemary, or tea tree can cost $4-$6, while a ⅓-ounce bottle of Turkish rose oil can cost $175. Thus, it is highly desirable that they be used with great efficiency, that is, that a high percentage of the material be taken up in the olfactory system of the consumer.

Further, such aromatic materials tend to evaporate readily and also to lose potency when subjected to sunlight. Therefore, they typically are stored in amber glass bottles to extend an otherwise short shelf life.

Aromatherapy requires a means for delivering the aromatic agent to the nose. In the prior art, a typical treatment can comprise from as little as a few sniffs of the agent to long sessions with several hundred sniffs per day. In general, however, a relatively long exposure to the aromatic material is most beneficial.

Such exposure in the prior art can be difficult and expensive to provide. Since the sensation of aroma may require only minute amounts of airborne aromatic material, full-strength materials may be diluted with innocuous carrier oils such as animal or mineral oils to form lotions or creams. The aromatic mixture may be rubbed onto the body around the neck and chest, where normal body heat vaporizes the mixture to release the aromatic material for inhalation. The person treated may have to wash the oil off the body after treatment to resume normal daily activities, otherwise the aroma can be detected by other persons throughout the day. Many persons experience skin irritation or allergic reactions to such topical application.

Alternatively, a mixture may be delivered by a diffuser vaporizer wherein the oils are mixed with water and heated to release the agent, or may be mixed into candle wax, candle wick, or incense stick and released by burning of the candle or stick. These vaporizing methods tend to provide aromatic agent throughout large areas, and thus are highly wasteful of the materials. Further, these methods are not practical for use while engaged in many other activities, such as while driving a car, riding on a bus, exercising in a gymnasium, or being outdoors or in an office or in other public places.

Another prior art delivery means is via bath oil, bath soap, and bath gel perfumes. To be therapeutic, these mixtures require the consumer to bathe, and such fragrances used on the body are noticed by other persons and may have to be separately washed off in order not to offend. To change the aroma several times per day, using this method, is impractical, and some aromatherapy fragrances, such as banana or peppermint, may not be desirable body fragrances.

Still other prior art delivery means are devices such as a wick or cotton ball saturated with aromatic oil and contained in a cylinder, ampule, or felt-tip container, whereby the aromatic material can be delivered to the nose by sniffing the device several times in each nostril throughout the day. While more efficient than the above-described means in delivering the material to the nose, these devices usually cannot be used discreetly in public, and further, require the use of two hands, one to hold the cap or to hold one nostril closed, and the other to administer the material. To use these devices, the consumer must stop an activity such as writing, typing, exercising, reading, or sleeping to administer the aromatherapy, which can be impractical especially when several hundred sniffs per day must be provided.

What is needed is a passive, unobtrusive device for continuous delivery of an aromatherapeutic agent to a consumer's olfactory system via normal breathing which is comfortable to use and is inexpensive to manufacture.

U.S. Pat. No. 5,850,834 discloses a U-shaped nose clip wherein opposed elements are magnetized to secured the clip onto a wearer's nasal septum by magnetic attraction of the elements. Because the thickness of the septum can vary among individual users, the strength of the magnetic attraction can be insufficient to retain the clip in some wearers' noses.

U.S. Pat. No. 5,479,944 discloses a flat nose clip which may be bent into a curved spring prior to insertion into a wearer's nostrils wherein it is retained by spring force against the nasal side walls. This mode of retention can be unpleasant for some persons.

It is a principal object of the invention to provide an improved device for continuously supplying an aroma-inducing agent directly into the nose of a consumer.

It is a further object of the invention to provide an improved supplying device which may be worn continuously without discomfort.

It is a still further object of the invention to provide an improved supplying device which may be worn inconspicuously.

It is a still further object of the invention to provide an improved supplying device which does not impinge the supplied aroma onto other persons, that is, which limits the supplied aroma to an individual consumer.

It is a still further object of the invention to provide an improved supplying device which has no moving parts and requires no source of power.

It is a still further object of the invention to provide an improved supplying device which is hygienic, does not absorb moisture, and can be washed as desired without loss in effectiveness.

It is a still further object of the invention to provide a array of improved supplying devices whereby an array of aromas can be presented quickly and efficiently.

It is a still further object of the invention to provide an improved supplying device which can be manufactured inexpensively.

Briefly described, an aroma delivery device in accordance with the invention comprises a horseshoe-shaped clip formed of a non-volatile elastomeric resin combined with one or more volatile aromatic materials, the clip being comfortably and easily applied to and retained on the cartilagineous septum of a wearer's nose, whereby the aromatic material being continuously given off by the clip is entrained in the nasal inhalations of a wearer and conveyed thereby to the wearer's olfactory sensors to create a desired aroma sensation.

Methods for combining the aromatic materials with elastomeric resin can include, but are not limited to, compounding the materials with the resin during formation of the resin, applying the materials to the surface of the clip after formation thereof, or, preferably, applying the materials to the surface of resin beads just prior to injection molding of the clip.

In a preferred embodiment of the invention, formed in accordance with the preferred method, the clip is permeated throughout by the aromatic materials such that emission of the materials is virtually undiminished over long periods of use, and further, that such emission is continued though the clip be cleaned, washed, reshaped, or resurfaced.

In a further preferred embodiment of the invention, a clip supplement including a second aromatic material is provided which may be mechanically attached to a clip including a first aromatic material, thereby affording a user a choice of combinations of secondary aromas with the primary clip aroma.

The foregoing and other objects, features, and advantages of the invention, as well as presently preferred embodiments thereof, will become more apparent from a reading of the following description in connection with the accompanying drawings in which:

FIG. 1 is a plan view of a prior art ornamental nasal clip;

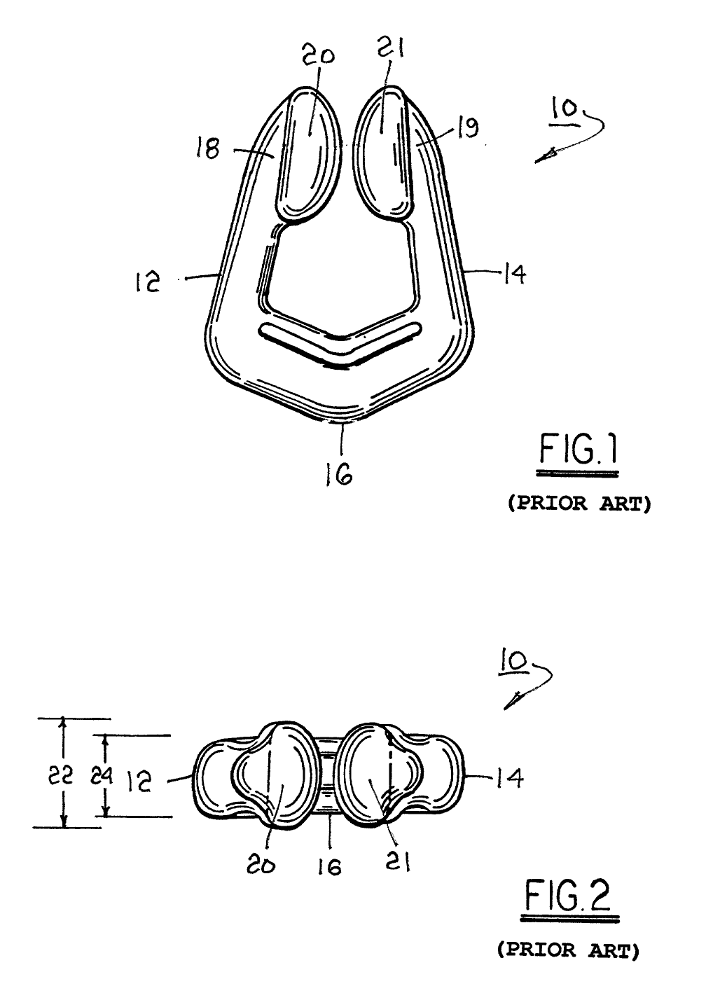

FIG. 2 is a front elevational view of the nasal clip shown in FIG. 1;

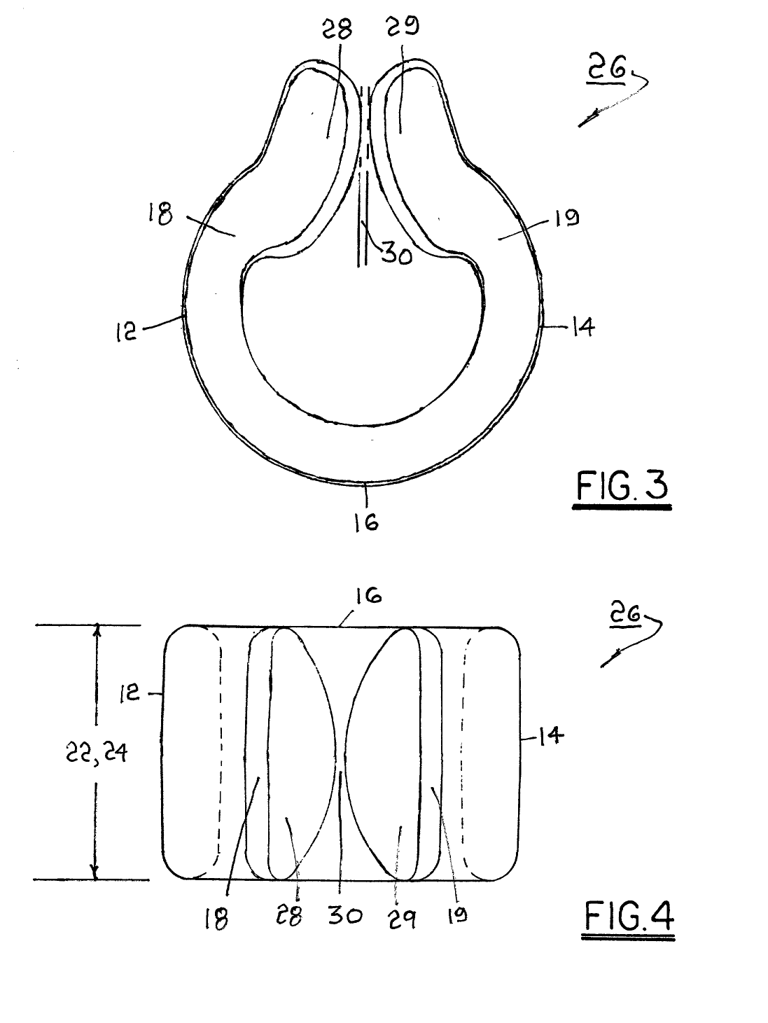

FIG. 3 is a plan view of a first preferred embodiment of an aroma delivery device in accordance with the invention;

FIG. 4 is an elevational view of the device shown in FIG. 3;

FIG. 5 is a plan view of a second preferred embodiment;

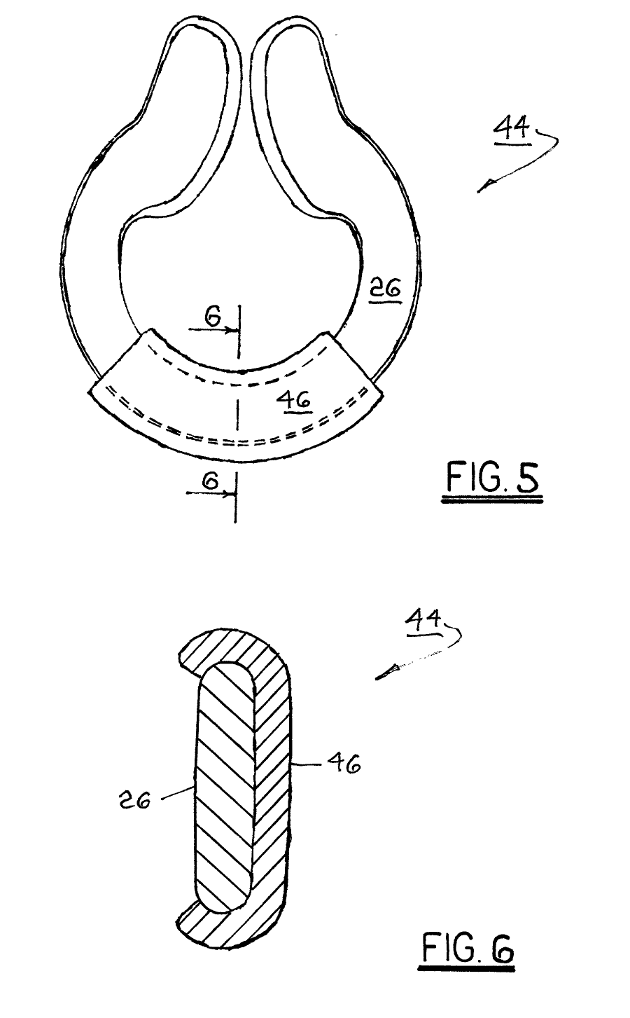

FIG. 6 is a cross-sectional view of the device shown in FIG. 5, taken along line 6-6 in FIG. 5;

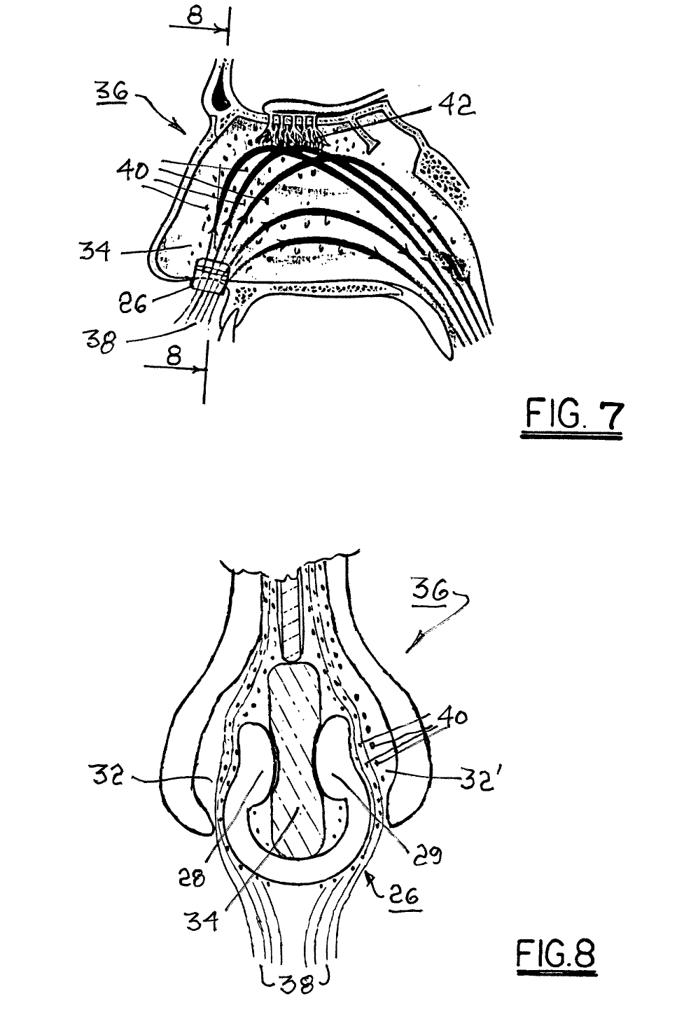

FIG. 7 is a first vertical cross-sectional view of the device shown in FIG. 3, showing the device installed in a wearer's nose, and showing the transfer of vaporized aromatic materials from the device to the wearer's olfactory sensors by inhaled breath; and

FIG. 8 is a second cross-sectional view orthogonal to the view shown in FIG. 7, taken along line 8-8 in FIG. 7.

Referring to FIGS. 1 and 2, prior art ornamental nasal clip 10 is shown substantially as in U.S. Design Pat. No. 375,552 issued Nov. 12, 1996 to Davi. As clip 10 is disclosed in a design patent, it has no stated utility but is similar in overall shape and size to an aroma delivery device in accordance with the invention. Clip 10 is generally V-shaped, comprising a first limb 12, second limb 14, and connector 16 connecting the first and second limbs. The free ends 18, 19 of each limb 12, 14, respectively, are provided with opposed non-spherical three-dimensionally arcuate portions 20, 21 each having a larger diameter 22 than the diameter 24 of either limb.

Referring to FIGS. 3, 4, 7, and 8, an aroma delivery device 26 in accordance with the invention has first and second limbs 12, 14 connected by connector 16 and having free ends 18, 19 thereof provided with opposed three-dimensional portions 28, 29 which are preferably partial cylindrical sections. The distance 30 between portions 28, 29 is preferably less than the thickness of the nasal septum of a wearer of the device, such that the device must be elastically deformed by pulling apart the opposed portions to engage the septum and is retained on the septum by the elastic force of the deformed device. The generally circular shape shown in these figures is presently preferred, although any shape wherein opposed portions engage opposite sides of the septum to retain the device in the nose is within the scope of the invention. Preferably, diameter 24 of the limbs is about the same as diameter 22 of opposed portions 28, 29.

Device 26 may be formed of any elastically-deformable material which is non-allergenic and has no significant odor, and preferably is medical grade, for example, polymeric resin such as polycarbonate, polyolefin, polybutyrate, polyethylene terephthalate, polyamide, polyimide, and polyvinyl chloride. Preferably, the forming material may be provided as resin beads which can be melted and extruded in a conventional extrusion/injection molding machine, as injection molding is a preferred method of making the device. The presently preferred material is medical grade polyvinyl chloride, provided as resin beads, for example, compound no. R1004-8803-C, available from Richlind Industries, Inc., Paramus, N.J. USA.

Device 26 includes an amount of one or more aromatic materials such as, for example, a plant or animal substance, essence, or other extract or naturally-occurring chemical or compound, or a synthetic chemical or compound. Preferably, such material is a liquid, food-grade fragrance supplied to fragrance industry standards as “Generally Regarded As Safe” (G.R.A.S.). This material may be added to the resin during or after the process of forming the polymer, or it may be coated onto device 26 after formation of the device, or it may be added in accordance with a presently preferred method of making device 26, wherein resin beads are conditioned with aromatic material prior to being introduced into the extruder hopper. A first amount of non-aromatic resin beads is combined with a second amount of aromatic material in a predetermined ratio. Preferably, the combination is tumbled in a tumble mill for about 20 minutes to distribute the aromatic material evenly over the surface of the beads. If the aromatic material does not mechanically adhere generally to the resin beads prior to the tumbling step, an amount of mineral oil may be added to promote satisfactory adhesion. After tumbling, the coated resin beads are added to the extruder hopper, and device 26 is injection molded conventionally in a female mold having the shape of the device. Different aromatic materials may have different volatilities, and thus more volatile materials may require lower extrusion temperatures to avoid significant loss of volatile materials during extrusion. Preferably, device 26 is cured for about 48 hours at room temperature after molding.

A suitable aromatic material, for examples is lavender scent, catalogue no. 3742-090G, available from Incredible Scents, Inc., Glen Head, N.Y. USA.

The preferred process just described is highly beneficial to the device manufacturer. The molder may vary the formulation ratio in small batches without having to inventory large quantities of pre-formulated beads. Further, shelf-life of large molding-ready batches is not a consideration, since each batch may be extruded immediately after tumbling. Further, manufacturing cost can be reduced over the cost of using pre-formulated resins which may require long lead times and be quite expensive. Instead, the molder can purchase relatively inexpensively the unformulated resin in large quantities and store it, if necessary, for long periods without risk of loss of aromatic components therefrom.

The completed device 26 comprises a three-dimensional polymeric structure fully permeated at the molecular level by the aromatic material. The encapsulated aromatic material is mobile within the polymeric structure and slowly outgasses therefrom with time. An aroma delivery device formed in this way may have a useful lifetime exceeding one years. Such outgassing takes place at all exposed surfaces of the device.

In operation, referring to FIGS. 7 and 8, opposed arcuate portions 28, 29 are retracted and device 26 is inserted into nostrils 32, 32′ with portions 28, 29 disposed on opposite sides of septum 34. Portions 28, 29 are released and immediately clasp septum 34 therebetween by spring force, thereby releasably retaining device 26 in nose 36. Removal is the reverse of installation. Preferably, diameters 22, 24 (FIG. 4) are small relative to the diameter of each nostril such that breathing is not significantly inhibited by the presence of device 26 in the nostrils.

Thus emplaced, device 26 is strategically located at the entrance point of the nasal inhalation air stream 38. Molecules 40 of aromatic material escaping from device 26 during inhalation are turbulently entrained in air stream 38, a significant portion of which is directed into contact with the mucous-coated epithelial receptors 42, where the sensation of aroma is initiated. Entrained molecules not received during inhalation can make contact with the receptors during exhalation, thus increasing the efficiency of the device.

Device 26 is non-allergenic, non-irritating, and may be left in place comfortably for as long as is desired by the wearer. It may be washed or otherwise cleaned without loss of function, and because the aromatic material is present throughout the volume of the preferred embodiment of the device, the original surfaces of the device may be trimmed or adjusted if desired without loss of outgassing area.

Referring to FIGS. 5 and 6, a second embodiment 44 of an aroma delivery device in accordance with the invention includes a supplementary element 46, which may also be formed by injection molding. Element 46 can readily be formulated to include one or more aromatic materials different from those included in device 26. Element 46 is attachable to device 26, and preferably is trough-shaped as shown in FIG. 6 to permit its being snapped onto device 26 as shown in FIG. 5. Thus, device 26 may be provided with a widely used aromatic material such as G.R.A.S. lavender which may be combined via various aromatic formulations of element 46 to provide lavender in combination with, for example, peppermint, clove, rosemary, chamomile, or geranium.

From the foregoing description, it will be apparent that there has been provided an improved aroma delivery device, wherein an elastomeric nasal clip carries and presents aromatic materials into the nasal cavity. Variations and modifications of the herein described aroma delivery devices in accordance with the invention, will undoubtedly suggest themselves to those skilled in this art. Accordingly, the foregoing description should be taken as illustrative and not in a limiting sense.

What is claimed is:

1. An aroma delivery device for delivering molecules of aromatic material into a wearer's nasal cavity, comprising a nasal clip containing an aromatic material for altering the mental state of a wearer, said clip including first and second opposed portions for releasably gripping the septum of a wearer's nose to retain said clip therein.

2. A device in accordance with claim 1 wherein said clip further includes first and second limbs for supporting said first and second opposed portions respectively, and a connector for connecting said limbs to form said clip.

3. A device in accordance with claim 2 wherein said clip is formed of a polymer resin selected form the group consisting of polycarbonate, polyolefin, polybutyrate, polyethylene terephthalate, polyamide, polyimide, and polyvinyl chloride.

4. A device in accordance with claim 3 wherein said aromatic material is selected from the group consisting of plant substance, plant essence, plant extract, animal substance, animal extract, and synthetic chemical.

5. A device in accordance with claim 4 wherein said aromatic material is a Generally Regarded As Safe fragrance.

6. A device in accordance with claim 1 further comprising a supplementary element attachable to said device for providing an additional aromatic material.

7. A method for making an aroma delivery nasal clip, comprising the steps of:
a) providing a female mold in the shape of said nasal clip;
b) providing a first amount of polymer resin beads;
c) providing a second amount of an aromatic material;
d) combining said first and second amounts;
e) liquefying said combination; and
f) injecting said liquefied combination into said mold.

8. A method in accordance with claim 7 comprising the further step of milling said combination of first and second amounts prior to said liquefying step.

9. An aroma delivery nasal clip formed by the method of claim 7.

10. A clip in accordance with claim 9 further comprising a supplementary element also formed by the method of claim 7.
